(19) United States
(12) Patent Application Publication
(10) Pub. No.: US 2007/0231870 A1
(43) Pub. Date: Oct. 4, 2007

(54) Process for the upgrading of heavy crude oil, extra-heavy crude oil or bitumens through the addition of a biocatalyst

(75) Inventors: Vladimir Leon, Caracas (VE); Jose Cordova, Caracas (VE); Spartacus Munoz, Caracas (VE); Angela De Sisto, Caracas (VE); Leopoldo Naranjo, Caracas (VE)

Correspondence Address: MERCHANT & GOULD PC, P.O. BOX 2903, MINNEAPOLIS, MN, 55402-0903, US

(73) Assignee: FUNDACION INSTITUTO DE ESTUDIOS AVANZADOS (IDEA), Caracas (VE)

(21) Appl. No.: 11/731,029

(22) Filed: Mar. 30, 2007

(30) Foreign Application Priority Data
Mar. 31, 2006 (VE) 00661-2006

(51) Int. Cl. C12P 5/00

(52) U.S. Cl. 435/166

(57) ABSTRACT

A process for the upgrading of heavy crude oil, extra-heavy crude oil or bitumens through the addition of a biocatalyst in the hydrocarbon flow, that includes the steps of: i) selecting the biological material for the preparation of the biocatalyst; ii) dispersing the biocatalyst in the flow of heavy crude, extra-heavy crude or bitumen; and iii) subjecting the crude oil mixture and biocatalyst to a process of bioconversion, separation, recovery and subsequent thermal process of the biotreated crude oil. The bioactive material and process to prepare the biocatalyst are moreover provided.

### BACKGROUND OF THE INVENTION

The invention refers to a process for the upgrading of heavy crude oil, extra-heavy crude oil or bitumen through the addition of a biocatalyst, as well as the process to prepare the biocatalyst.

Several processes are known to upgrade heavy crude oil, extra-heavy crude oil or bitumen in light hydrocarbons. These processes include visbreaking and coking at extreme temperatures (delayed coking). Nevertheless, these processes are characterized by the low rates of conversion and/or a high percentage of waste products, such as coke, which among others produced problems of transport, handling and disposal of wastes. Also, the use of disperse catalysts has been described in the patent literature for the upgrading of heavy crude oil and residual oil. For example, U.S. Pat. No. 6,043,182 (Córdova et al., 2000) describes the dispersion of catalytic materials in heavy crude oil or residuals for their subsequent upgrading in processes of hydroconversion, vaporconversion, visbreaking, coking, among others. Moreover, in U.S. Pat. No. 5,885,441 (Pereira et al., 1999) the dispersion of catalysts in heavy crude oil and residual oil is described form their subsequent upgrading with steam. Both patents were developed to upgrade heavy crude oil, extra-heavy crude oil or bitumens.

Moreover, in the previous art the use of microorganisms on crude oil has been reported (for example: U.S. Pat. No. 5,858,866, Premuzic et al., 1999). The potentiality of the use of the microorganism is proposed in the paper published by V. León and M. Kumar (2005).

The invention developed starts from the analysis of the individual characteristics of the deposits of Venezuelan heavy crude oil, extra-heavy crude oil or bitumens, located in the oil belt of the Orinoco and the need to develop additives and technology that allow the improvement of the processes of extraction, transport and commercialization thereof.

The oil belt of the Orinoco is a large sedimentary basin, located north of the Orinoco River, which covers an extension of 600 km east-west and 70 km north-south, with an area of approximately 55,000 Km2. It is the principal deposit of heavy crude oil and extra-heavy crude oil or bitumens in the world. The size of the deposit is approximately 1.5 trillion barrels, and considering a possible extraction of 20% through the use of current technology, would mean some proven reserves of 300 billion barrels.

In the ^{o}API (American Petroleum Institute) gravity scale, extra-heavy crude oil or bitumens are located in the range of 8-12^{o} API and the heavy crude oil in the range of 12-20^{o} API. These crude oils are characterized by having a high viscosity, with high concentrations of asphaltenes, resins, heteroatoms (S, N, O) and metals (nickel and vanadium). Currently, the production of crude oil in the oil belt of the Orinoco reaches about 600,000 barrels per day, whereby synthetic crude oil, generated exclusively for thermal processes, are produced.

The purpose of this invention is to provide a method for the upgrading of heavy crude oil, extra-heavy crude oil or bitumen through the addition of a biocatalyst in the hydrocarbon flow, as well as provide the bioactive material and the process for the preparation of this biocatalyst.

The additive and process disclosed in this invention permits modifying the physical and chemical properties of density and viscosity of the heavy crude oil, extra-heavy crude oil or bitumens, increasing the quantity and quality of the distillates and reducing the corresponding vacuum residual. Moreover, this technology can be directly employed in the well head and in a subsequent phase, it can permit the direct commercialization of upgrades crude oil.

Other purposes and advantages of this invention will be indicated as shown below:

### SUMMARY OF THE INVENTION

As the invention indicates, the following purposes and advantages are obtained through the application of the process and biocatalyst that are claimed.

The invention claims a process for the biochemical conversion of a heavy crude oil, extra-heavy crude or bitumen flow in presence of a biocatalyst. This process includes the stages of: (i) selecting the biological material for the preparation of the biocatalyst; ii) dispersing the biocatalyst in the flow of heavy crude oil, extra-heavy crude oil or bitumen, and (iii) subjecting the mixture of crude oil and biocatalyst to a process of bioconversion, separation, recovery and subsequent thermal process of the biotreated crude oil, in order to provide upgraded crude oil. The bioactive material and the process to prepare the biocatalyst are moreover provided.

### BRIEF DESCRIPTION OF THE DRAWINGS

A detailed description of the characteristics of the invention is indicated below, with reference to the drawings that are attached, where:

FIG. 1: This is a schematic representation of the process of biochemical conversion related to this invention, for the production of an upgraded crude oil starting from heavy crude oil, extra-heavy crude oil or bitumens.

FIG. 2: This is a schematic representation of the process of biochemical conversion related to this invention.

### DEPOSIT

A number of microorganisms of use in the process of this invention (Table 1 and 2) have been deposited in the Centro Venezolano de Colección de Microorganismos (CVCM) before the presentation of this application. Below, the microorganisms deposited are listed:

TABLE I
IDENTIFICATION AND CHARACTERIZATION OF AUTOCHTHONOUS
BACTERIA ABLE TO METABOLIZE HEAVY CRUDE OIL, EXTRA-HEAVY
CRUDE OIL OR BITUMEN

| No. | Stains- | | | | | | | |
|---|---|---|---|---|---|---|---|---|
| CVCM2 | Molecular ID | Nomenclature | Source | Naphthalene | Fenantrene | Pirene | DBT | INDOL |
| 1750 | Acinetobacter | AN-01B | Anzoategui | − | − | + | − | − |
| | Radiore | | | | | | | |
| | Mirandastens | | | | | | | |
| 1751 | Acinetobacter | CCDI | Aragua | − | − | + | − | − |
| | Radiore | | | | | | | |
| | Mirandastens | | | | | | | |
| 1752 | Ochrobactrum | CMA | Miranda | ++ | + | + | + | + |
| | intermedium | | | | | | | |
| 1753 | Comamonas | CMN | Miranda | ++ | + | + | − | + |
| | testosteroni | | | | | | | |
| 1754 | Ochrobactrum | C1BM2B | Miranda | − | − | − | − | − |
| | intermedium | | | | | | | |
| 1755 | Acinetobacter | G10A | Sucre | − | − | + | − | − |
| | Baumanii | | | | | | | |
| 1756 | Bacilus | G10B | Sucre | − | − | + | . | . |
| | afuMirandaformis | | | | | | | |
| 1757 | To be | G11D1 | Sucre | ++ | + | − | + | + |
| | identified | | | | | | | |
| 1758 | Comamonas | G11D2 | Sucre | ++ | + | + | + | + |
| | testosterone | | | | | | | |
| 1759 | Comamonas | G11Q | Sucre | ++ | + | − | + | + |
| | testosterone | | | | | | | |
| 1760 | Comamonas | G12A | Sucre | − | − | − | − | − |
| | testosterone | | | | | | | |
| 1761 | Comamonas | G13Z2 | Sucre | ++ | + | + | + | + |
| | testosterone | | | | | | | |
| 1762 | Comamonas | G14X | Sucre | ++ | + | − | + | + |
| | testosterone | | | | | | | |
| 1763 | Chryseobacterium | G20Y | Sucre | ++ | − | + | − | − |
| | formosense | | | | | | | |
| 1764 | Comamonas | G30DN | Sucre | ++ | + | − | + | + |
| | testosterone | | | | | | | |
| 1765 | Comamonas | G30ZN | Sucre | ++ | + | − | + | + |
| | testosterone | | | | | | | |
| 1766 | Comamonas | I1 | Miranda | − | − | − | − | − |
| | testosterone | | | | | | | |
| 1767 | Pseudomonas | R1 | Miranda | ++ | − | + | + | + |
| | putida/Pseudomonas | | | | | | | |
| | piecoglosssicida | | | | | | | |
| 1768 | Acinetobacter | BM3 | Sucre | − | − | + | − | − |
| | baumannii | | | | | | | |
| 1769 | Staphylocossus | BM1A | Sucre | − | − | − | − | − |
| | pasteuri | | | | | | | |
| 1770 | Providencia | G5AD | Sucre | − | − | − | − | − |
| | stuartii | | | | | | | |
| 1771 | To be | G5CG | Sucre | − | − | − | − | − |
| | identified | | | | | | | |
| 1772 | Ochorbactrum | G5CP | Sucre | ++ | − | − | − | + |
| | intermedium | | | | | | | |
| 1774 | Pseudomonas | 5a1 | Sucre | ++ | + | + | + | + |
| | putida/Pseudomonas | | | | | | | |
| | fulva | | | | | | | |
| 1775 | Gamma | EP02A | Sucre | ++ | + | − | + | + |
| | protobacterium | | | | | | | |
| 1776 | Pseudomonas | SAM1 | Miranda | ++ | − | + | + | + |
| | putida/Pseudomonas | | | | | | | |
| | fulva | | | | | | | |
| 1777 | Pseudomonas | DH-1 | Sucre | | | | | |
| | aeruginosa | | | | | | | |
| 1778 | Pseudomonas | DH-2 | Sucre | | | | | |
| | thermaerum/ | | | | | | | |
| | Pseudomonas | | | | | | | |
| | aeruginosa | | | | | | | |
| 1779 | Pseudomonas | DH-3 | Sucre | | | | | |
| | aeruginosa | | | | | | | |
| 1780 | Pseudomonas | DH-4 | Sucre | | | | | |
| | aeruginosa | | | | | | | |
| 1781 | Providencia | DHT-5 | Sucre | | | | | |
| | rettgeri | | | | | | | |
| 1782 | Pseudomonas | DH-6 | Sucre | | | | | |
| | pseudoalcaligenes | | | | | | | |
| 1783 | Bacillus | DH-7 | Sucre | | | | | |
| | lichenformis | | | | | | | |

TABLE 2

| | Strain | | Minimum Czapek medium with PAHs as only source of carbon and energy | | | | | | | | | | | | | |
|---|---|---|---|---|---|---|---|---|---|---|---|---|---|---|---|---|
| No. | (classical | | | | | | | | | | | | | | Hexane | |
| CVCM1 | & mole. ID | Surce | Control+ | | Control− | | Pirene | | Fanantrene | | DBT | | Naphthalene | | cycle | |
| 1784 | Fusarium | Sucre | +++ | +++ | +/− | +/− | +++ | +++ | ++ | ++ | ++ | ++ | +++ | +++ | + | + |
| | solani HP-1 | | | | | | | | | | | | | | | |
| 1785 | Fusarium | Sucre | +++ | +++ | − | − | + | + | ++ | ++ | ++ | ++ | ++ | ++ | ++ | ++ |
| | proliferatum | | | | | | | | | | | | | | | |
| 1786 | Cladosporium | Sucre | ++ | ++ | − | − | + | + | + | + | + | + | + | + | * | * |
| | sphaerospermum | | | | | | | | | | | | | | | |
| 1787 | Pestalotiposis | Sucre | +++ | +++ | − | − | + | + | + | + | + | + | + | + | * | * |
| | sp. | | | | | | | | | | | | | | | |
| 1788 | Neosartorya | Sucre | +++ | +++ | − | − | ++ | ++ | + | + | + | + | ++ | ++ | + | − |
| | sp. 1 | | | | | | | | | | | | | | | |
| 1789 | Neosartorya | Sucre | +++ | +++ | − | − | + | + | ++ | ++ | ++ | ++ | ++ | ++ | + | + |
| | sp. 2 | | | | | | | | | | | | | | | |
| 1790 | Penicillium | Sucre | +++ | +++ | +/− | +/− | ++ | ++ | +/− | +/− | +/− | +/− | + | + | ++ | ++ |
| | sp. 1 | | | | | | | | | | | | | | | |
| 1791 | Fusarium | Miranda | +++ | +++ | +/− | +/− | + | + | + | + | + | + | + | + | ++ | ++ |
| | sp. 1 | | | | | | | | | | | | | | | |
| 1792 | Fusarium | Miranda | +++ | +++ | + | + | + | + | + | + | + | + | + | + | ++ | ++ |
| | sp. 2 | | | | | | | | | | | | | | | |
| 1793 | Penicillium | Miranda | ++ | ++ | +/− | +/− | ++ | ++ | + | + | + | + | ++ | ++ | ++ | ++ |
| | sp. 2 | | | | | | | | | | | | | | | |
| 1794 | Aspergilus | Miranda | +++ | +++ | − | − | + | + | + | + | + | + | + | + | ++ | ++ |
| | aff. | | | | | | | | | | | | | | | |
| | Fumigatus | | | | | | | | | | | | | | | |
| 1795 | Trichoderma | Miranda | +++ | +++ | − | − | + | + | + | + | − | − | + | + | ++ | ++ |
| | sp. 1 | | | | | | | | | | | | | | | |
| 1796 | Trichoderma | Miranda | +++ | +++ | − | − | + | + | + | + | + | + | +/− | +/− | ++ | ++ |
| | sp. 2 | | | | | | | | | | | | | | | |
| 1797 | Penisillium | Miranda | +++ | +++ | − | − | + | + | − | − | + | + | ++ | ++ | ++ | ++ |
| | sp. 3 | | | | | | | | | | | | | | | |
| 1798 | Trichoderma | Miranda | +++ | +++ | − | − | ++ | ++ | ++ | ++ | ++ | ++ | ++ | ++ | + | + |
| | sp. 3 | | | | | | | | | | | | | | | |
| 1799 | Aspergillus | Miranda | +++ | +++ | − | − | ++ | ++ | ++ | ++ | ++ | ++ | ++ | ++ | ++ | ++ |
| | aff. | | | | | | | | | | | | | | | |
| | Terreus | | | | | | | | | | | | | | | |
| 1800 | Penicillium | Miranda | ++ | ++ | − | − | ++ | ++ | ++ | ++ | ++ | ++ | ++ | ++ | ++ | ++ |
| | sp. 4 | | | | | | | | | | | | | | | |
| 1801 | Paqecillomyces | Miranda | +++ | +++ | +/− | +/− | ++ | ++ | ++ | ++ | ++ | ++ | ++ | ++ | ++ | ++ |
| | sp. | | | | | | | | | | | | | | | |
| 1802 | Fusarium | Miranda | +++ | +++ | + | + | + | + | + | + | + | + | + | + | ++ | ++ |
| | sp. 3 | | | | | | | | | | | | | | | |
| 1803 | Fusarium | Miranda | +++ | +++ | − | − | + | + | ++ | ++ | + | + | − | − | ++ | * |
| | sp. 4 | | | | | | | | | | | | | | | |

1 Access number: Centro Venezolano de Coleccion de Micoorganismos (CVCM)
*Characterization not made of cyclohexane. + Slight growth. ++ Moderate growth. +++ Good growth.

### DETAILED DESCRIPTION OF THE INVENTION

This invention provides a new process for the upgrading of heavy crude oil, extra-heavy crude oil or bitumens, through the biochemical conversion of the crude oil produced by the addition of a biocatalyst, which consists of the use of live microorganisms (bacteria or fungi) or their enzymatic extracts. The process can be applied, whether at the well head to obtain an upgraded crude that can even be directly commercialized, or sent to a thermal process with additional advantages, which permits increasing the quantity and quality of the distillates and reduce the corresponding vacuum residual. According to the invention, the flow is contacted under conditions of biochemical conversion, through the action of a biocatalyst in the aqueous phase of an emulsion of a heavy crude oil, extra-heavy crude oil or bitumen.

The biochemical conditions of conversion according to this invention include: a temperature from 9° C. to 90° C., preferably in the range of from 30° C. to 60° C.; an air pressure less than or equal to 150 psi, and preferably from 15 psi to 150 psi, ideally less than or equal to 75 psi and preferably from 10 psi to 150 psi; a reaction time from 3 hours to 72 hours (bioconversion) depending on the severity of the desired treatment and under aerobic conditions.

In a second stage, and after separating the biotreated crude oil, it is subjected to a thermal process with the following characteristics: temperature from 260° C. to 530° C., preferably from 350° C. to 470° C. and a reaction time from 1 to 4 hours depending on the severity of the desired treatment.

The conditions of bioconversion are more advantageous compared with the conventional conversion with hydrogen, because lower pressures than those required to maintain the hydrogenation can be used. Thus, the process of biochemical conversion of this invention permits the reduction of costs of equipment and operating costs, derived from conditions at high temperatures, producing at the same time a lighter crude oil, with the advantage that its elementary composition in carbons and hydrogens and its calorific power are maintained.

In the distillations of the crude oil biotreated at the scale of laboratory, simulating atmospheric and vacuum distillations, an improvement in the performances of the distillates of the biotreated crude oils in respect to the control crude oil, which in this case is the Cerro Negro extra-heavy crude oil, extracted from the Oil Belt of the Orinoco, was found. Table 3 below contains the characteristics of a typical flow of crude oil that can serve as example, for the purposes of the application of the process of this invention.

This invention permits, among other biochemical process, the oxidation of the aromatic carbon atoms, weakening the carbon-carbon bonds. Then, if a thermal process is subsequently incorporated, the output of the current technology is improved, since at 200° C. the expected effect is produced.

The subsequent thermal process at 350° C. and for one hour reports the improvement in the output that is appreciated in Tables 6 and 7, demonstrating that the thermal process is necessary to appreciate the significant improvement of this invention in the treatment of the heavy crude oil, extra-heavy crude oil or bitumens.

In the initial test, under coking conditions, the change was observed of the molecules of the crude oil, on obtaining a distillate of 24^{o} API for the control crude oil and 36^{o} API for the biotreated crude oil, even achieving 10% of desulfurization, as indicated in Table 5.

The presence of water is a fundamental factor for the operation of the invention, so that emulsions on site are prepared that increase the water-crude interaction. The emulsions, up to the size of a drop of one micron (1 μm), permit upgrading the interface area of contact, which is necessary, because the invention involves the use of microorganisms or their enzymes, which are soluble in water. The crude-water proportion of the emulsion, as a working range for this invention is from 30:70 to 70:30.

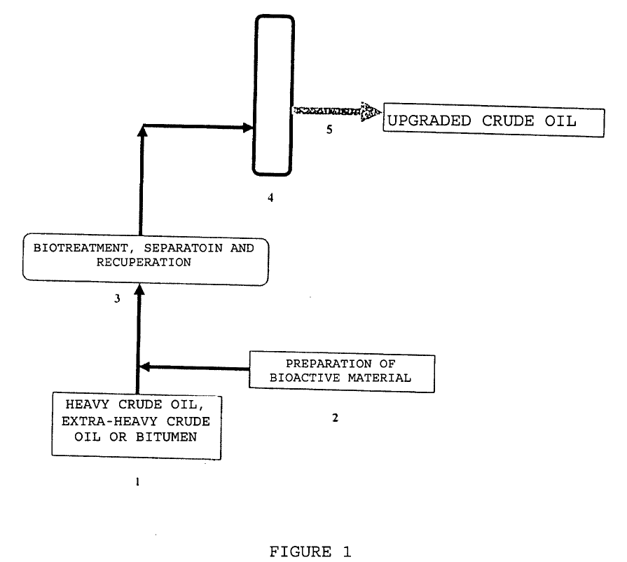

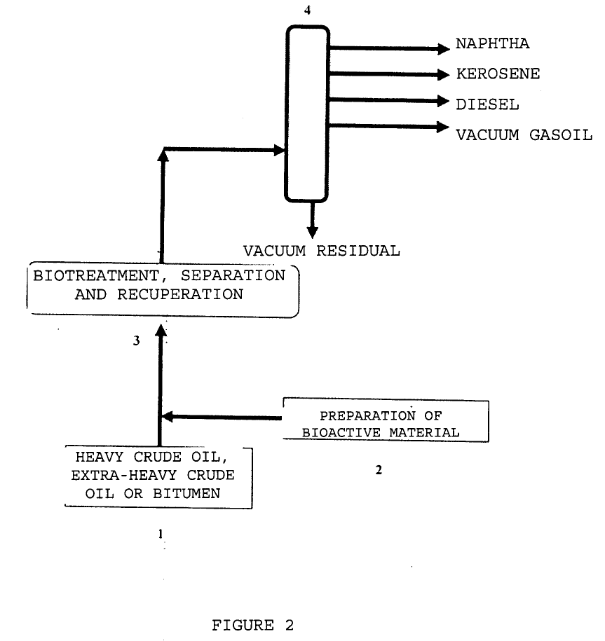

The biocatalyst and process disclosed in this invention allow modifying the physical and chemical properties of density and viscosity of the heavy crude oil, extra-heavy crude oil or bitumen, upgrading the distillates and reducing the corresponding vacuum residual. Moreover, this technology can be directly employed in well head and in a subsequent phase it can permit the direct commercialization of the improved crude oils, as detailed in FIGS. 1 and 2.

In FIG. 1, the heavy crude oil, extra-heavy crude oil or bitumen (1) is dispersed with aqueous phase of microorganisms and/or their enzymes (2) and passes to the biotreatment reactor in which the bioconversion and subsequent separation of the biotreated crude oil (3) occurs. Finally, and after the dispersion is separated, the biotreated crude oil is subjected to thermal treatment (4) to produce upgraded crude oil (5).

In FIG. 2, the heavy crude oil, extra-heavy crude oil or bitumen (1) is dispersed with aqueous phase of microorganisms and/or their enzymes (2) and passes to the biotreatment reactor in which the bioconversion and subsequent separation of the biotreated crude (3) occurs. Finally, and after the dispersion is separated, the biotreated crude passes to the breaker (4) where upgraded products are separated into several factions, including naphtha, kerosene, diesel, gasoil and vacuum residual.

It is important to signal that FIGS. 1 and 2 are simply schematic representations of the configurations of the process of this invention, and it is understood that the process can be carried out with variations of the steps and with different equipment, including without limitation, the use of any thermal process in the refinery or downstream with operating temperatures equal to or greater than 350° C.

According to this invention, a biocatalyst is provided that is adopted for bioconversion of heavy crude oil, extra-heavy crude oil or bitumens to convert them into upgraded crude oil. The biocatalyst that is claimed involves at least a bacteria or its enzymatic extracts, a consortium of bacteria or their enzymatic extracts, or a fungus and its enzymatic extracts, or a consortium of funguses or their enzymatic extracts; selected from the group indicated in Table 1 or in Table 2 and prepared according to the methodology that is indicated below:

### Bacterial Consortium

The bacterial strains selected were inoculated separately in a minimum M9 medium (Piddington et al., 1995), supplemented with naphthalene as the only source of carbon and energy and were incubated in an orbital shaker at 30° C. and 200 rpm for 24 hours, until reaching the late exponential growth phase (titer: 1×10⁹ UFC/ml). Subsequently, the fresh cells were recovered by centrifugation at 5000 rpm for 15 minutes at 4° C., resuspended in 2.5 ml of medium M9 and were mixed to obtain a final volume of 12.5 ml. The mentioned mixture (bacterial consortium) constituted the biocatalyst agent. The bioconversion in vivo, carried out in a volume of 100 ml contained in 500 ml flasks, was initiated inoculating a mixture made up of 87.5 ml of en emulsion of Cerro Negro extra-heavy crude, oil/water (O/W) 54:46, prepared in M9, with 12.5 ml of bacterial consortium, at the end 100 ml of emulsion of oil/water (O/W) of approximately 50:50 being obtained.

For the case of a single bacterium, the procedure described in similar, scaling the corresponding volumes.

### Bacterial Cellular Extracts

In this case, each bacterium was inoculated separately in a minimum M9 medium with naphthalene as the only source of carbon and energy and incubated at 30° C. and 200 rpm for 24 hours until reaching the late growth exponential phase (titer: 1×10⁹ UFC/ml). The cells were recuperated by centrifugation at 5000 rpm for 30 minutes at 4° C. and were resuspended in Na—K buffer (50 mM, pH 7.4). The cellular extracts were obtained by sonication. The cellular remains were discarded by centrifugation, 1.5 ml of each supernatant were taken and mixed to obtain a final volume of 7.5 ml. This mixture of cellular extracts constituted the biocatalyst agent.

The bioconversion with the bacterial cellular extracts was carried out in a volume of 100 ml contained in 500 ml flasks, was initiated mixing 92.5 ml of an emulsion of the Cerro Negro extra-heavy crude oil/water (O/W) 51:49, prepared in M9, with 7.5 ml of the mixture of bacterial cellular extracts, obtaining at the end 100 ml of an oil/water (O/W) emulsion of approximately 50:50.

For the case of a single bacterial strain, the procedure described is similar, scaling the corresponding volumes.

### Fungi Biocatalyst

Spores of a fungus selected from Table 2 are obtained, grown for 10 days at 30° C. in plates with Czapek minimum medium (Naranjo et al., 2001) supplemented with 1% of an emulsion of Cerro Negro extra-heavy crude. Fresh spores of the fungus selected were used to inoculate 100 ml of Czapek medium supplemented with naphthalene as only source of carbon and energy, and they were incubated at 30° C. and 250 rpm for 96 hours.

The bioconversion in vivo with the fungus biocatalyst was carried out in a volume of 100 ml contained in 500 ml flasks, was initiated mixing 80 ml of an emulsion of the Cerro Negro extra-heavy crude oil/water (O/W) 60:40, prepared in Czapek medium with carbon source; and 20 ml of the inoculate of the selected fungus was added to it, obtaining at the end 100 ml of an oil/water (O/W) emulsion of approximately 50:50.

For the case of a consortium of fungi, the procedure described is similar, scaling the corresponding volumes.

### Fungus Cellular Extracts

Spores of a fungus selected from Table 2 are obtained, grown for 10 days at 30° C. in plates with Czapek minimum medium (Naranjo et al., 2001) supplemented with 1% of an emulsion of Cerro Negro extra-heavy crude. Fresh spores of the fungus selected were used to inoculate 100 ml of Czapek medium supplemented with naphthalene as only source of carbon and energy, and they were incubated at 30° C. and 250 rpm for 96 hours.

The bioconversion with the fungus cellular extracts was carried out in a volume of 100 ml contained in 500 ml flasks, was initiated mixing 80 ml of an emulsion of the Cerro Negro extra-heavy crude oil/water (O/W) 60:40, prepared in Czapek medium with carbon source; and 20 ml of the enzymatic extracts of the selected fungus, prepared in Na—K buffer, was added to it, obtaining at the end 100 ml of an oil/water (O/W) emulsion of approximately 50:50.

For the case of enzymatic extracts consortium of fungi, the procedure described is similar, scaling the corresponding volumes.

### Advantages of this Invention

The advantages of this invention, mentioned previously, are observed in the examples that follow below:

Table 3 below indicates some of the physical and chemical characteristics of the Cerro Negro extra-heavy crude oil:

TABLE 3

| | |
|---|---|
| Carbon (% wt) | 80.3 |
| Hydrogen (% wt) | 9.9 |
| Sulfur (% wt) | 3.7 |
| Nitrogen (% wt) | 0.6 |
| Metals (ppm) | 462 |
| Gravity °API | 8.4 |
| Asphaltenes (% wt) | 11.5 |
| Conradson Carbon (% wt) | 17.2 |

### EXAMPLE 1

In this example, as indicated in Table 4, the advantages of this invention are illustrated when the distillates obtained by coking of the control Cerro Negro extra-heavy crude oil are compared with those obtained from the crude oil biotreated through the process of this invention.

TABLE 4

| | | Distillates of the |
|---|---|---|
| | Distillates of the | control Cerro |
| | biotreated crude | Negro crude |
| °API | 29 | 24 |
| Sulfur (% wt) | 2.99 | 3.22 |
| Nitrogen (%) | 0.26 | 0.29 |

This example corresponds to the process with the bacteria No. CVCM 1774. Both the biotreatment flask and that of control, without the biocatalyst agent, were incubated in an orbital shaker at 30° C. and 200 rpm. Samples of 20 ml were collected at various fermentation times (16, 24 and 72 hours). The emulsion was broken at 90-100° C., the recovered crude oil was washed with distilled water and then they were both subjected to coking.

### EXAMPLE 2

This example, as Table 5 indicates, illustrates the process of this invention executed with the Consortium of degrading authocthonous bacterial strains of extra-heavy crude oil No. CVCN 1776, 1777, 1774, 1752 and 1753. Both the biotreatment flask and that of the control, without the biocatalyst agent, were incubated in an orbital shaker at 30° C. and 200 rpm. Samples of 20 ml were collected at various fermentation times (16, 24 and 72 hours). The emulsion was broken at 90-100° C., the recovered crude oil was washed with distilled water and then they were both subjected to coking.

TABLE 5

| | | Distillates of the |
|---|---|---|
| | Distillates of the | control Cerro |
| | biotreated crude | Negro crude |
| °API | 36 | 24 |
| Sulfur (% wt) | 2.99 | 3.22 |
| Nitrogen (%) | 0.26 | 0.29 |

This example, as indicated in Table 6, illustrates the advantages of the process of this invention executed with the enzymatic extract of the consortium described in example 2, through the simulated distillation of the crude oils obtained.

TABLE 6

| Distillation (% | | Cerro Negro |
|---|---|---|
| wt) | Biotreated crude | Control crude |
| Naphtha, IBP-200° C. | 0.94 | 0.56 |
| Jet, 200–250° C. | 2.86 | 0.59 |
| Diesel, 250–350° C. | 12.45 | 9.05 |
| GOV, 350–500° C. | 27.37 | 23.37 |
| RV, 500° C.+ | 56.38 | 66.99 |

Both the biotreatment flask and that of the control (without the biocatalyst agent) were incubated in an orbital shaker at 30° C. and 200 rpm. Samples of 20 ml were collected at various fermentation times (16, 24 and 72 hours). The emulsion was broken at 90-100° C., the recovered crude oil was washed with distilled water and then they were both subjected to a thermal process at 350° C. for one hour.

As shown, the advantages of this invention are evident on the basis of the excellent conversion of the fraction of residual 500° C.+, and the corresponding high performance upgraded light hydrocarbons.

### EXAMPLE 4

This example, as indicated in Table 7, illustrates the advantages of the process of this invention using the fungus No. CVCM 1784, through the simulated distillation of the crude oils obtained.

TABLE 7

| | | Cerro Negro |
|---|---|---|
| | Biotreated crude | Control crude (% |
| Distillation | (% wt) | wt) |
| Naphtha, IBP-200° C. | 1.41 | 1.08 |
| Jet, 200–250° C. | 2.13 | 2.23 |
| Diesel, 250–350° C. | 14.46 | 8.80 |
| GOV, 350–500° C. | 28.54 | 28.63 |
| RV, 500° C.+ (% wt) | 53.46 | 60.34 |

Both the biotreatment flask and that of the control (without the biocatalyst agent) were incubated at 30° C. and 250 rpm and samples were taken at 24, 48, 72, 96 and 120 hours of fermentation. Then, the emulsion was broken at 90-100° C., the recovered crude oil was washed with distilled water and then they were both subjected to a thermal process at 350° C. for one hour.

As shown, the advantages of this invention are evident on the basis of the excellent conversion of the fraction of residual 500° C.+, and the corresponding high performance of upgraded light hydrocarbons.

This invention can have other configurations or be carried out in other manners, without becoming removed from the spirit of this invention or the essential characteristics thereof. This configuration must be considered as a consequence to be illustrative of the invention and not restrictive of the scope thereof indicated in the claims, and all the changes that are within the meaning or range of equivalence are included therein.

What is claimed is:

1. A method for upgrading heavy crude oils, extra-heavy crude oils or bitumens through the action of a biocatalyst in the hydrocarbon flow, comprising:
a. selecting the biological material from the group consisting of
naphthalene negative, phenanthrene negative, pyrene positive, dibenzothiophene (DBT) negative, and indol negative Acinetobacter radioresistens;
naphthalene positive, phenanthrene positive, pyrene positive, DBT positive, and indol positive Ochrobactrum intermedium;
naphthalene positive, phenanthrene positive, pyrene positive, DBT negative, and indol positive Comamonas testosteroni;
naphthalene negative, phenanthrene negative, pyrene negative, DBT negative, and indol negative Ochrobactrum intermedium;
naphthalene negative, phenanthrene negative, pyrene positive, DBT negative, and indol negative Acinetobacter baumannii;
naphthalene negative, phenanthrene negative, pyrene positive, DBT negative, and indol negative Bacillus fusiformis;
naphthalene positive, phenanthrene positive, pyrene positive, DBT positive, and indol positive Comamonas testosteroni;
naphthalene positive, phenanthrene positive, pyrene negative, DBT positive, and indol positive Comamonas testosteroni;
naphthalene negative, phenanthrene negative, pyrene negative, DBT negative, and indol negative Comamonas testosteroni;
naphthalene positive, phenanthrene negative, pyrene positive, DBT negative, and indol negative Chryseobacterium formosense;
naphthalene positive, phenanthrene negative, pyrene positive, DBT positive, and indol positive Pseudomonas putida; naphthalene positive, phenanthrene negative, pyrene positive, DBT positive, and indol positive Pseudomonas plecoglossicida;
naphthalene negative, phenanthrene negative, pyrene positive, DBT negative, and indol negative Acinetobacter baumannii;
naphthalene negative, phenanthrene negative, pyrene negative, DBT negative, and indol negative Staphylococcus pasteuri;
naphthalene negative, phenanthrene negative, pyrene negative, DBT negative, and indol negative Providencia stuartii;
naphthalene positive, phenanthrene negative, pyrene negative, DBT negative, and indol negative Ochrobactrum intermedium;
naphthalene positive, phenanthrene positive, pyrene positive, DBT positive, and indol positive Pseudomonas putida;
naphthalene positive, phenanthrene positive, pyrene positive, DBT positive, and indol positive Pseudomonas fulva;
naphthalene positive, phenanthrene positive, pyrene negative, DBT positive, and indol positive Gamma proteobacterium;
naphthalene positive, phenanthrene negative, pyrene positive, DBT positive, and indol positive Pseudomonas putida; naphthalene positive, phenanthrene negative, pyrene positive, DBT positive, and indol positive Pseudomonas fulva;
thermotolerant and surfactant producing Pseudomonas aeruginosa; Pseudomonas thermaerum; Providencia rettgeri; Pseudomonas pseudoalcaligenes; and Bacillus licheniformis;
and/or pyrene positive, phenanthrene positive, DBT positive, naphthalene positive, cyclohexane positive Fusarium solani;
pyrene positive, phenanthrene positive, DBT positive, naphthalene positive, cyclohexane positive Fusarium proliferatum;
pyrene positive, phenanthrene positive, DBT positive, naphthalene positive Cladosporium sphaerospermum;
pyrene positive, phenanthrene positive, DBT positive, naphthalene positive Pestalotiopsis sp.;
pyrene positive, phenanthrene positive, DBT positive, naphthalene positive, cyclohexane positive Neosartorya sp. 1;
pyrene positive, phenanthrene positive, DBT positive, naphthalene positive, cyclohexane positive Neosartorya sp. 2;
pyrene positive, naphthalene positive, cyclohexane positive Penicillium sp. 1;
pyrene positive, phenanthrene positive, DBT positive, naphthalene positive, cyclohexane positive Fusarium sp. 1;
pyrene positive, phenanthrene positive, DBT positive, naphthalene positive, cyclohexane positive Fusarium sp. 2;
pyrene positive, phenanthrene positive, DBT positive, naphthalene positive, cyclohexane positive Penicillium sp. 2;
pyrene positive, phenanthrene positive, DBT positive, naphthalene positive, cyclohexane positive Aspergillus aff. fumigatus;
pyrene positive, phenanthrene positive, DBT negative, naphthalene positive, cyclohexane positive Trichoderma sp. 1;
pyrene positive, phenanthrene positive, DBT positive, cyclohexane positive Trichoderma sp. 2;
pyrene positive, DBT positive, naphthalene positive, cyclohexane positive Penicillium sp. 3;
pyrene positive, phenanthrene positive, DBT positive, naphthalene positive, cyclohexane positive Trichoderma sp. 3;
pyrene positive, phenanthrene positive, DBT positive, naphthalene positive, cyclohexane positive Aspergillus aff. terreus;
pyrene positive, phenanthrene positive, DBT positive, naphthalene positive, cyclohexane positive Penicillium sp. 4;
pyrene positive, phenanthrene positive, DBT positive, naphthalene positive, cyclohexane positive Paecillomyces sp.;
pyrene positive, phenanthrene positive, DBT positive, naphthalene positive, cyclohexane positive Fusarium sp. 3;
pyrene positive, phenanthrene positive, DBT positive, naphthalene negative, cyclohexane positive Fusarium sp.4
for the preparation of the biocatalyst;
b. dispersing the biocatalyst in the flow of heavy crude oils, extra-heavy crude oils or bitumen;
c. subjecting the crude mixture and biocatalyst to a bioconversion process;
d. breaking the emulsion and separating the biotreated crude; and
e. submitting the biotreated crude to a thermal process, in order to provide upgraded crude oils.

2. The method of claim 1, where the step of selecting the bacterial biological material for the preparation of the biocatalyst from the group consisting of
naphthalene negative, phenanthrene negative, pyrene positive, dibenzothiophene (DBT) negative, and indol negative Acinetobacter radioresistens;
naphthalene positive, phenanthrene positive, pyrene positive, DBT positive, and indol positive Ochrobactrum intermedium;
naphthalene positive, phenanthrene positive, pyrene positive, DBT negative, and indol positive Comamonas testosteroni;
naphthalene negative, phenanthrene negative, pyrene negative, DBT negative, and indol negative Ochrobactrum intermedium;
naphthalene negative, phenanthrene negative, pyrene positive, DBT negative, and indol negative Acinetobacter baumannii;
naphthalene negative, phenanthrene negative, pyrene positive, DBT negative, and indol negative Bacillus fusiformis;
naphthalene positive, phenanthrene positive, pyrene positive, DBT positive, and indol positive Comamonas testosteroni;
naphthalene positive, phenanthrene positive, pyrene negative, DBT positive, and indol positive Comamonas testosteroni;
naphthalene negative, phenanthrene negative, pyrene negative, DBT negative, and indol negative Comamonas testosteroni;
naphthalene positive, phenanthrene negative, pyrene positive, DBT negative, and indol negative Chryseobacterium formosense;
naphthalene positive, phenanthrene negative, pyrene positive, DBT positive, and indol positive Pseudomonas putida; naphthalene positive, phenanthrene negative, pyrene positive, DBT positive, and indol positive Pseudomonas plecoglossicida;
naphthalene negative, phenanthrene negative, pyrene positive, DBT negative, and indol negative Acinetobacter baumannii;
naphthalene negative, phenanthrene negative, pyrene negative, DBT negative, and indol negative Staphylococcus pasteuri;
naphthalene negative, phenanthrene negative, pyrene negative, DBT negative, and indol negative Providencia stuartii;
naphthalene positive, phenanthrene negative, pyrene negative, DBT negative, and indol negative Ochrobactrum intermedium;
naphthalene positive, phenanthrene positive, pyrene positive, DBT positive, and indol positive Pseudomonas putida;
naphthalene positive, phenanthrene positive, pyrene positive, DBT positive, and indol positive Pseudomonas fulva;
naphthalene positive, phenanthrene positive, pyrene negative, DBT positive, and indol positive Gamma proteobacterium;
naphthalene positive, phenanthrene negative, pyrene positive, DBT positive, and indol positive Pseudomonas putida; naphthalene positive, phenanthrene negative, pyrene positive, DBT positive, and indol positive Pseudomonas fulva;
thermotolerant and surfactant producing Pseudomonas aeruginosa; Pseudomonas thermaerum; Providencia rettgeri; Pseudomonas pseudoalcaligenes; and Bacillus licheniformis;
comprises:
a. selecting at least one bacterial strain with capacity to degrade polyaromatic molecules and heavy crude oils, extra-heavy crude oils or bitumen;
b. growing the bacterial strains separately in the M9 minimum culture medium supplemented with naphthalene as only source of carbon and energy; and
c. recovering the cells and mixing them in M9 minimum culture.

3. The method of claim 1, where the step of selecting the bacterial biological material for the preparation of the biocatalyst from the group consisting of
naphthalene negative, phenanthrene negative, pyrene positive, dibenzothiophene (DBT) negative, and indol negative Acinetobacter radioresistens;
naphthalene positive, phenanthrene positive, pyrene positive, DBT positive, and indol positive Ochrobactrum intermedium;
naphthalene positive, phenanthrene positive, pyrene positive, DBT negative, and indol positive Comamonas testosteroni;
naphthalene negative, phenanthrene negative, pyrene negative, DBT negative, and indol negative Ochrobactrum intermedium;
naphthalene negative, phenanthrene negative, pyrene positive, DBT negative, and indol negative Acinetobacter baumannii;
naphthalene negative, phenanthrene negative, pyrene positive, DBT negative, and indol negative Bacillus fusiformis;
naphthalene positive, phenanthrene positive, pyrene positive, DBT positive, and indol positive Comamonas testosteroni;
naphthalene positive, phenanthrene positive, pyrene negative, DBT positive, and indol positive Comamonas testosteroni;
naphthalene negative, phenanthrene negative, pyrene negative, DBT negative, and indol negative Comamonas testosteroni;
naphthalene positive, phenanthrene negative, pyrene positive, DBT negative, and indol negative Chryseobacterium formosense;
naphthalene positive, phenanthrene negative, pyrene positive, DBT positive, and indol positive Pseudomonas putida; naphthalene positive, phenanthrene negative, pyrene positive, DBT positive, and indol positive Pseudomonas plecoglossicida;
naphthalene negative, phenanthrene negative, pyrene positive, DBT negative, and indol negative Acinetobacter baumannii;
naphthalene negative, phenanthrene negative, pyrene negative, DBT negative, and indol negative Staphylococcus pasteuri;
naphthalene negative, phenanthrene negative, pyrene negative, DBT negative, and indol negative Providencia stuartii;
naphthalene positive, phenanthrene negative, pyrene negative, DBT negative, and indol negative Ochrobactrum intermedium;
naphthalene positive, phenanthrene positive, pyrene positive, DBT positive, and indol positive Pseudomonas putida;
naphthalene positive, phenanthrene positive, pyrene positive, DBT positive, and indol positive Pseudomonas fulva;
naphthalene positive, phenanthrene positive, pyrene negative, DBT positive, and indol positive Gamma proteobacterium;
naphthalene positive, phenanthrene negative, pyrene positive, DBT positive, and indol positive Pseudomonas putida; naphthalene positive, phenanthrene negative, pyrene positive, DBT positive, and indol positive Pseudomonas fulva;
thermotolerant and surfactant producing Pseudomonas aeruginosa; Pseudomonas thermaerum; Providencia rettgeri; Pseudomonas pseudoalcaligenes; and Bacillus licheniformis;
comprises:
a. selecting at least one bacterial strain with capacity to degrade polyaromatic molecules and heavy crude oils, extra-heavy crude oils or bitumen;
b. growing the bacterial strains separately in the M9 minimum culture medium supplemented with naphthalene as only source of carbon and energy; and
c. preparing the bacterial enzymatic extracts separately and then mixing them in Na—K buffer.

4. The method of claim 1, where the step of selecting the fungi biological material for the preparation of the biocatalyst from the group consisting of pyrene positive, phenanthrene positive, DBT positive, naphthalene positive, cyclohexane positive Fusarium solani;
pyrene positive, phenanthrene positive, DBT positive, naphthalene positive, cyclohexane positive Fusarium proliferatum;
pyrene positive, phenanthrene positive, DBT positive, naphthalene positive Cladosporium sphaerospermum;
pyrene positive, phenanthrene positive, DBT positive, naphthalene positive Pestalotiopsis sp.;
pyrene positive, phenanthrene positive, DBT positive, naphthalene positive, cyclohexane positive Neosartorya sp. 1;
pyrene positive, phenanthrene positive, DBT positive, naphthalene positive, cyclohexane positive Neosartorya sp. 2;
pyrene positive, naphthalene positive, cyclohexane positive Penicillium sp. 1;
pyrene positive, phenanthrene positive, DBT positive, naphthalene positive, cyclohexane positive Fusarium sp. 1;
pyrene positive, phenanthrene positive, DBT positive, naphthalene positive, cyclohexane positive Fusarium sp. 2;
pyrene positive, phenanthrene positive, DBT positive, naphthalene positive, cyclohexane positive Penicillium sp. 2;
pyrene positive, phenanthrene positive, DBT positive, naphthalene positive, cyclohexane positive Aspergillus aff. fumigatus;
pyrene positive, phenanthrene positive, DBT negative, naphthalene positive, cyclohexane positive Trichoderma sp. 1;
pyrene positive, phenanthrene positive, DBT positive, cyclohexane positive Trichoderma sp. 2;
pyrene positive, DBT positive, naphthalene positive, cyclohexane positive Penicillium sp. 3;
pyrene positive, phenanthrene positive, DBT positive, naphthalene positive, cyclohexane positive Trichoderma sp. 3;
pyrene positive, phenanthrene positive, DBT positive, naphthalene positive, cyclohexane positive Aspergillus aff. terreus;
pyrene positive, phenanthrene positive, DBT positive, naphthalene positive, cyclohexane positive Penicillium sp. 4;
pyrene positive, phenanthrene positive, DBT positive, naphthalene positive, cyclohexane positive Paecillomyces sp.;
pyrene positive, phenanthrene positive, DBT positive, naphthalene positive, cyclohexane positive Fusarium sp. 3;
pyrene positive, phenanthrene positive, DBT positive, naphthalene negative, cyclohexane positive Fusarium sp. 4
comprises:
a. selecting at least one fungus strain with capacity to degrade polyaromatic molecules and heavy crude oils, extra-heavy crude oils or bitumen; and
b) inoculating fresh spores of the selected fungus in the Czapek minimum culture medium supplemented with naphthalene as only source of carbon and energy to obtain the inoculate that will constitute the biocatalyst.

5. The method of claim 1, where the step of selecting the fungi biological material for the preparation of the biocatalyst from the group consisting of CVCM No. pyrene positive, phenanthrene positive, DBT positive, naphthalene positive, cyclohexane positive Fusarium solani;
pyrene positive, phenanthrene positive, DBT positive, naphthalene positive, cyclohexane positive Fusarium proliferatum;
pyrene positive, phenanthrene positive, DBT positive, naphthalene positive Cladosporium sphaerospermum;
pyrene positive, phenanthrene positive, DBT positive, naphthalene positive Pestalotiopsis sp.;
pyrene positive, phenanthrene positive, DBT positive, naphthalene positive, cyclohexane positive Neosartorya sp. 1;
pyrene positive, phenanthrene positive, DBT positive, naphthalene positive, cyclohexane positive Neosartorya sp. 2;
pyrene positive, naphthalene positive, cyclohexane positive Penicillium sp. 1;
pyrene positive, phenanthrene positive, DBT positive, naphthalene positive, cyclohexane positive Fusarium sp. 1;
pyrene positive, phenanthrene positive, DBT positive, naphthalene positive, cyclohexane positive Fusarium sp. 2;
pyrene positive, phenanthrene positive, DBT positive, naphthalene positive, cyclohexane positive Penicillium sp. 2;
pyrene positive, phenanthrene positive, DBT positive, naphthalene positive, cyclohexane positive Aspergillus aff. fumigatus;
pyrene positive, phenanthrene positive, DBT negative, naphthalene positive, cyclohexane positive Trichoderma sp. 1;
pyrene positive, phenanthrene positive, DBT positive, cyclohexane positive Trichoderma sp. 2;
pyrene positive, DBT positive, naphthalene positive, cyclohexane positive Penicillium sp. 3;
pyrene positive, phenanthrene positive, DBT positive, naphthalene positive, cyclohexane positive Trichoderma sp. 3;
pyrene positive, phenanthrene positive, DBT positive, naphthalene positive, cyclohexane positive Aspergillus aff. terreus;
pyrene positive, phenanthrene positive, DBT positive, naphthalene positive, cyclohexane positive Penicillium sp. 4;
pyrene positive, phenanthrene positive, DBT positive, naphthalene positive, cyclohexane positive Paecillomyces sp.;
pyrene positive, phenanthrene positive, DBT positive, naphthalene positive, cyclohexane positive Fusarium sp. 3;
pyrene positive, phenanthrene positive, DBT positive, naphthalene negative, cyclohexane positive Fusarium sp. 4
comprises:
a. selecting at least one fungus strain with capacity to degrade polyaromatic molecules and heavy crude oils, extra-heavy crude oils or bitumen;
b) inoculating fresh spores of the selected fungus in the Czapek minimum culture medium supplemented with naphthalene as only source of carbon and energy; and
c) preparing the bacterial enzymatic extracts separately and then mixing them in Na—K buffer.

6. The method of claim 1, where the biocatalyst is dispersed in aqueous phase of a heavy crude oil, extra-heavy crude oil or bitumen emulsion.

7. The method of claim 6, where the emulsion is of the oil/water (O/W) type in a proportion of water no greater than 70%.

8. The method of claim 6, in which the solution of the biocatalyst is diluted in the aqueous phase of the emulsion.

9. The method of claim 6, in which the biocatalyst is dispersed to directly conform an emulsion at well head, maintaining water in a proportion no greater than 50%.

10. The method of claim 2, where the biocatalyst in the aqueous phase comprises a titer no greater than 10 times the titer reached in the late exponential growth phase.

11. The method of claim 3, where the concentration of the enzymatic extract of the biocatalyst in the aqueous phase is up to 10 times greater than the concentration obtained form the titer reached in the late exponential growth phase.

12. The method of claim 4, where the fungus biocatalyst in the aqueous phase comprises a growth phase no greater than 96 hours.

13. The method of claim 5, where the concentration of the enzymatic extract of the fungus biocatalyst in the aqueous phase is up to 10 times greater than the concentration obtained from a culture grown up to 96 hours.

14. The method of claim 1, where the bioconversion occurs at a temperature of 9° C. to 70° C.

15. The method of claim 1, where the bioconversion occurs during a reaction time of 3 to 72 hours.

16. The method of claim 1, where the bioconversion occurs at an air pressure from 10 psi to 150 psi.

17. The method of claim 1, where the biotreated crude is separated breaking the emulsion through a convention crude-water separation process.

18. The method of claim 17, where the separated biotreated crude is recovered through a conventional process.

19. The method of claim 1, where the biotreated crude oil is subjected to any thermal process installed in the refinery or any other process established downstream, with operating temperature equal to or greater than 350° C.

20. The method of claim 1, whereby after the thermal process an upgraded crude oil is obtained.
